(19) United States
(12) Patent Application Publication
(10) Pub. No.: US 2023/0050258 A1
(43) Pub. Date: Feb. 16, 2023

(54) IMMUNOTHERAPEUTIC TREATMENT OF CANCER

(71) Applicant: IMMODULON THERAPEUTICS LIMITED, LONDON (GB)

(72) Inventor: GLEN MARTYN, LEICESTERSHIRE (GB)

(21) Appl. No.: 17/758,139

(22) PCT Filed: Dec. 29, 2020
(86) PCT No.: PCT/GB2020/053375

§ 371 Date: Jun. 29, 2022

(30) Foreign Application Priority Data
Dec. 30, 2019 (GB) 1919428.1

(51) Int. Cl. A61K 35/741; A61K 9/00; A61K 39/395; A61K 35/768; A61K 38/22; A61M 37/00

(52) U.S. Cl. CPC A61K 35/741; A61K 9/0021; A61K 39/3955; A61K 35/768; A61K 38/2292; A61M 37/0015; A61M 2037/0061; A61M 2037/0023

(57) ABSTRACT

The present invention relates to the field of cancer therapy. In particular, the present invention relates to a method of preventing, treating or inhibiting the development of tumours and/or metastases in a subject by administering one or more immunomodulators into the skin via a microneedle device.

### FIELD OF THE INVENTION

The present invention relates to the field of cancer therapy. In particular, the present invention relates to a method of preventing, treating or inhibiting the development of tumours and/or metastases in a subject by administering one or more immunomodulators into the skin via a microneedle device.

### BACKGROUND OF THE INVENTION

In humans with advanced cancer, anti-tumour immunity becomes ineffective due to the tightly regulated interplay of pro- and anti-inflammatory, immune-stimulatory and immunosuppressive signals. For example, loss of the anti-inflammatory signals leads to chronic inflammation and prolonged proliferative signalling. Interestingly, cytokines that both promote and suppress proliferation of the tumour cells are produced at the tumour site. It is the imbalance between the effects of these various processes that results in tumour promotion.

To date, a major barrier to attempts to develop effective immunotherapy for cancer has been an inability to break immunosuppression at the cancer site and restore normal networks of immune reactivity. The physiological approach of immunotherapy is to normalize the immune reactivity so that, for example, the endogenous tumour antigens would be recognized and effective cytolytic responses would be developed against tumour cells. Although it was once unclear if tumour immunosurveillance existed, it is now believed that the immune system constantly monitors and eliminates newly transformed cells. Accordingly, cancer cells may alter their phenotype in response to immune pressure in order to escape attack (immunoediting) and upregulate expression of inhibitory signals. Through immunoediting and other subversive processes, primary tumour and metastasis maintain their own survival.

However, producing effective treatment vaccines has proven to be challenging. To be effective, cancer treatment vaccines must achieve two goals. First, they must stimulate specific immune responses against the correct target and, second, the immune responses must be powerful enough to overcome the barriers that cancer cells use to protect themselves from attack by immune cells.

Transdermal delivery is severely limited by the inability of the large majority of drugs to cross skin at therapeutic rates given the great barrier imposed by skin's outer stratum corneum (SC) layer. Chemical/lipid enhancers, electric fields using iontophoresis and electroporation, and pressure waves generated by ultrasound or photo acoustic effects are some of different approaches to increase permeability of skin have been studied. Although the mechanisms are all different, these methods share the common goal of disrupting the SC structure to create “holes” big enough for molecules to pass through. The size of disruptions generated by each of these methods is believed to be of nanometer dimensions, which are large enough to permit transport of small drugs and, in some cases, macromolecules but probably small enough to prevent damage of clinical significance.

In the context of therapy based on administration of Mycobacteria via injection, there is a problem in that conventional injection techniques require training and may not consistently deliver the Mycobacteria to the same region and/or depth. Therefore, there can be a lack of effective and consistent targeting to the lymphatic vessels and/or dendritic cells and consequently a lack of induction of effective anti-tumour Type 1 responses. Moreover, there may also be skin reactions at the site of injection which may reduce future compliance.

There is therefore a need for improved administration of Mycobacteria through and into the skin.

### SUMMARY OF THE INVENTION

The present invention provides an effective method for treating and/or preventing cancer and/or the establishment of metastases by administering a whole cell, non-viable Mycobacterium into the skin via a microneedle device including at least one microneedle.

For the avoidance of doubt, the subject to be treated is preferably not a checkpoint inhibitor refractory patient.

In a first aspect of the invention, there is an immunomodulator for use in the treatment, reduction, inhibition or control of a neoplasia, tumour or cancer in a subject, wherein the immunomodulator comprises a non-pathogenic, non-viable Mycobacterium and wherein said immunomodulator is to be administered into the skin of said subject via a microneedle device comprising a plurality of microneedles.

In a second aspect of the invention, there is method of treating, reducing, inhibiting or controlling a neoplasia, tumour or cancer in a subject, and wherein said method comprises:
(i) providing a microneedle device comprising a plurality of microneedles,
(ii) causing the microneedles to penetrate through a stratum corneum into the skin of the subject and optionally assume an anchored state in which the microneedles are anchored in the skin, and project from the microneedle device,
(iii) delivering into the skin via the microneedle device, preferably subcutaneously, subdermally or intradermally, a quantity of a non-pathogenic non-viable Mycobacterium selected from M. vaccae, including the strain deposited under accession number NCTC 11659 and associated designations such as SRL172, SRP299, IMM-201, DAR-901, and the strain as deposited under ATCC 95051 (Vaccae™); M. obuense, M. paragordonae (strain 49061), M. parafortuitum, M. aurum, M. phlei, M. indicus pranii, M.w, M. kyogaense (as deposited under DSM 107316/CECT 9546), M. tuberculosis Aoyama B or H37Rv, and combinations thereof, preferably the strain of Mycobacterium obuense deposited under the Budapest Treaty under accession number NCTC 13365.

In a third aspect of the invention, there is method of treating, reducing, inhibiting or controlling a neoplasia, tumour or cancer in a subject, and wherein said method comprises:
(i) providing a microneedle device comprising a plurality of microneedles,
(ii) causing the microneedles to penetrate through a stratum corneum into the skin of the subject and optionally assume an anchored state in which the microneedles are anchored in the skin, and project from the microneedle device,
(iii) delivering into the skin via the microneedle device, preferably subcutaneously, subdermally or intradermally, a quantity of a non-pathogenic non-viable Mycobacterium,
wherein said method further comprises simultaneously, separately or sequentially administering to the subject, one or more biologically-active agents selected from a therapeutic drug, nutraceutical, vaccine, cell, virus, lysate, vector, gene, mRNA, DNA, nucleic acid, protein, polypeptide, peptide, antibody, bispecific antibody, multi-specific antibody, ADC (antibody-drug conjugate), Fab fragment (Fab), F(ab')2 fragment, diabody, triabody, tetrabody, probody, single-chain variable region fragment (scFv), disulfide-stabilized variable region fragment (dsFv), or other antigen binding fragment thereof, optionally wherein said biologically-active agent is an antigen or antigenic determinant.

In a fourth aspect of the invention, there is provided a method of treating, reducing, inhibiting or controlling a neoplasia, tumour or cancer in a subject, and wherein said method comprises:
(i) providing a microneedle device comprising a plurality of microneedles,
(ii) causing the microneedles to penetrate through a stratum corneum into the skin of the subject and optionally assume an anchored state in which the microneedles are anchored in the skin, and project from the microneedle device,
(iii) delivering into the skin via the microneedle device, preferably subcutaneously, subdermally or intradermally, a quantity of a non-pathogenic non-viable Mycobacterium selected from M. vaccae, including the strain deposited under accession number NCTC 11659 and associated designations such as SRL172, SRP299, IMM-201, DAR-901, and the strain as deposited under ATCC 95051 (Vaccae™); M. obuense, M. paragordonae (strain 49061), M. parafortuitum, M. aurum, M. phlei, M. indicus pranii, M.w, M. kyogaense (as deposited under DSM 107316/CECT 9546), M. tuberculosis Aoyama B or H37Rv, and combinations thereof, preferably the strain of Mycobacterium obuense deposited under the Budapest Treaty under accession number NCTC 13365,
wherein said method further comprises simultaneously, separately or sequentially administering to the subject, one or more biologically-active agents wherein the one or more biologically-active agents is a checkpoint inhibitor directed against CTLA-4, PD-1, PD-L1, PD-L2, B7-H3, B7-H4, B7-H6, A2AR, IDO, TIM-3, BTLA, VISTA, TIGIT, LAG-3, CD40, KIR, CEACAM1, GARP, PS, CSF1R, CD94/NKG2A, TDO, TNFR, DcR3 and combinations thereof.

In a fifth aspect of the invention, there is provided a kit of parts for delivering a non-pathogenic, whole-cell Mycobacterium into the skin of a subject, comprising:
a microneedle device comprising a plurality of microneedles, and,
a non-pathogenic non-viable Mycobacterium is selected from M. vaccae, including the strain deposited under accession number NCTC 11659 and associated designations such as SRL172, SRP299, IMM-201, DAR-901, and the strain as deposited under ATCC 95051 (Vaccae™); M. obuense, M. paragordonae (strain 49061), M. parafortuitum, M. aurum, M. phlei, M. indicus pranii, M.w, M. kyogaense (as deposited under DSM 107316/CECT 9546), M. tuberculosis Aoyama B or H37Rv, and combinations thereof, preferably the strain of Mycobacterium obuense deposited under the Budapest Treaty under accession number NCTC 13365, and;
one or more biologically-active agents selected from a therapeutic drug, nutraceutical, vaccine, cell, virus, lysate, vector, gene, mRNA, DNA, nucleic acid, protein, polypeptide, peptide, antibody, bispecific antibody, multi-specific antibody, ADC (antibody-drug conjugate), Fab fragment (Fab), F(ab')2 fragment, diabody, triabody, tetrabody, probody, single-chain variable region fragment (scFv), disulfide-stabilized variable region fragment (dsFv), or other antigen binding fragment thereof, optionally wherein said biologically-active agent is an antigen or antigenic determinant.

In a sixth aspect of the invention, there is provided a kit of parts for delivering a non-pathogenic, whole-cell Mycobacterium into the skin of a subject, comprising:
a microneedle device comprising a plurality of microneedles, and,
a non-pathogenic non-viable Mycobacterium is selected from M. vaccae, including the strain deposited under accession number NCTC 11659 and associated designations such as SRL172, SRP299, IMM-201, DAR-901, and the strain as deposited under ATCC 95051 (Vaccae™); M. obuense, M. paragordonae (strain 49061), M. parafortuitum, M. aurum, M. phlei, M. indicus pranii, M.w, M. kyogaense (as deposited under DSM 107316/CECT 9546), M. tuberculosis Aoyama B or H37Rv, and combinations thereof, preferably the strain of Mycobacterium obuense deposited under the Budapest Treaty under accession number NCTC 13365, and;
one or more biologically-active agents wherein said one or more biologically-active agents is a checkpoint inhibitor, selected from a cell, protein, peptide, antibody or antigen binding fragment thereof, directed against CTLA-4, PD-1, PD-L1, PD-L2, LAG-3, B7-H3, B7-H4, B7-H6, A2AR, IDO, TIM-3, BTLA, VISTA, TIGIT, LAG-3, CD40, KIR, CEACAM1, GARP, PS, CSF1R, CD94/NKG2A, TDO, TNFR, DcR3, CD27, CD28, CD40, CD122, CD137, 0X40, GITR, ICOS and combinations thereof.

According to a seventh aspect, there is a microneedle device comprising a plurality of microneedles, and contained thereon or therein a composition comprising a non-pathogenic, non-viable Mycobacterium.

### DETAILED DESCRIPTION OF THE INVENTION

The present invention provides an effective means to treat neoplastic disorders with immunotherapy via a microneedle device.

In order that the present invention may be more readily understood, certain terms are first defined. Additional definitions are set forth throughout the detailed description.

The terms “tumour,” “cancer” and “neoplasia” are used interchangeably and refer to a cell or population of cells whose growth, proliferation or survival is greater than growth, proliferation or survival of a normal counterpart cell, e.g. a cell proliferative or differentiative disorder. Typically, the growth is uncontrolled. The term “malignancy” refers to invasion of nearby tissue. The term “metastasis” refers to spread or dissemination of a tumour, cancer or neoplasia to other sites, locations or regions within the subject, in which the sites, locations or regions are distinct from the primary tumour or cancer.

The term “therapeutically effective amount” is defined as an amount of immunomodulator, optionally in combination with a whole cell, non-viable Mycobacterium, that preferably results in a decrease in severity of disease symptoms, an increase in frequency and duration of disease symptom-free periods, or a prevention of impairment or disability due to the disease affliction. The terms “effective amount” or “pharmaceutically effective amount” refer to a sufficient amount of an agent to provide the desired biological or therapeutic result. That result can be reduction, amelioration, palliation, lessening, delaying, and/or alleviation of one or more of the signs, symptoms, or causes of a disease, or any other desired alteration of a biological system. In reference to cancer, an effective amount may comprise an amount sufficient to cause a tumour to shrink and/or to decrease the growth rate of the tumour (such as to suppress tumour growth) or to prevent or delay other unwanted cell proliferation. In some embodiments, an effective amount is an amount sufficient to delay development, or induce stabilisation of the cancer or tumour. It may also prolong survival.

A “checkpoint inhibitor” is an agent which acts on surface proteins which are members of either the TNF receptor or B7 superfamilies, including agents which bind to negative co-stimulatory molecules selected from CTLA-4, PD-1, PD-L1, PD-L2, LAG-3, B7-H3, B7-H4, B7-H6, A2AR, IDO, TIM-3, BTLA, VISTA, TIGIT, LAG-3, CD40, KIR, CEACAM1, GARP, PS, CSFIR, CD94/NKG2A, TDO, TNFR, DcR3, CD27, CD28, CD40, CD122, CD137, 0X40, GITR, ICOS and combinations thereof, and/or their respective ligands, including PD-L1. (Mellman et al., supra). A “blocking agent” is an agent which either binds to the above co-stimulatory molecules and/or their respective ligands. “Checkpoint inhibitor” and “blocking agent” are used interchangeably throughout. The inhibitor is preferably an antibody or antigenic-binding molecule that targets an antigenic site on the surface proteins. For example, the inhibitor is an antibody that targets an antigenic site on PD-L1, or PD-1 or CTLA-4.

An immunomodulator, as defined according to the present invention, is a component which stimulates innate and type-1 immunity, including Th1 and macrophage activation and cytotoxic cell activity, as well as independently down-regulating inappropriate anti-Th2 responses via immunoregulatory mechanisms.

As used herein, “sub-therapeutic dose” means a dose of a therapeutic compound (e.g., an antibody) or duration of therapy which is lower than the usual or typical dose of the therapeutic compound or therapy of shorter duration, when administered alone for the treatment of cancer.

In some embodiments, a therapeutically effective amount is an amount sufficient to prevent or delay recurrence. A therapeutically effective amount can be administered in one or more administrations. The therapeutically effective amount of one or more immunomodulators or combinations described herein, may result in one or more of the following: (i) reduce the number of cancer cells; (ii) reduce tumour size; (iii) inhibit, retard, slow to some extent and preferably stop cancer cell infiltration into peripheral organs; (iv) inhibit (i.e., slow to some extent and preferably stop) tumour metastasis; (v) inhibit tumour growth; (vi) prevent or delay occurrence and/or recurrence of tumour; and/or (vii) relieve to some extent one or more of the symptoms associated with the cancer.

For example, for the treatment of tumours, a “therapeutically effective dosage” may induce tumour shrinkage by at least about 5% relative to baseline measurement, such as at least about 10%, or about 20%, or about 60% or more. The baseline measurement may be derived from untreated subjects.

A therapeutically effective amount of one or more immunomodulators can decrease tumour size, or otherwise ameliorate symptoms in a subject. One of ordinary skill in the art would be able to determine such amounts based on such factors as the subject's size, the severity of the subject's symptoms, and the particular composition or route of administration selected.

The term “immune response” refers to the action of, for example, lymphocytes, antigen presenting cells, phagocytic cells, granulocytes, and soluble macromolecules produced by the above cells or the liver (including antibodies, cytokines, and complement) that results in selective damage to, destruction of, or elimination from the human body of cancerous cells.

The term “antibody” as referred to herein includes whole antibodies and any antigen-binding fragment (i.e., “antigen-binding portion”) or single chains thereof.

The term “antigen-binding portion” of an antibody (or simply “antibody portion”), as used herein, refers to one or more fragments of an antibody that retain the ability to specifically bind to a receptor and its ligand (e.g., PD-1). including:(i) a Fab fragment, (ii) a F(ab') 2 fragment, (iii) a Fd fragment consisting of the VH and CHI domains; (iv) a Fv fragment, (v) a dAb fragment (Ward et al, Nature, 341 :544-546 (1989)), which consists of a VH domain; and (vi) an isolated complementarity determining region (CDR). Single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “human antibody”, as used herein, is intended to include antibodies having variable regions in which both the framework and CDR regions are derived from human germline immunoglobulin sequences.

The term “humanized antibody” is intended to refer to antibodies in which CDR sequences derived from the germline of another mammalian species, such as a mouse, have been grafted onto human framework sequences. Additional framework region modifications may be made within the human framework sequences.

In addition to antibodies, other biological molecules may act as checkpoint inhibitors, including peptides having binding affinity to the appropriate target.

The term “treatment” or “therapy” refers to administering an active agent with the purpose to cure, heal, alleviate, relieve, alter, remedy, ameliorate, improve, or affect a condition (e.g., a disease), the symptoms of the condition, or to prevent or delay the onset of the symptoms, complications, biochemical indicia of a disease, or otherwise arrest or inhibit further development of the disease, condition, or disorder in a statistically significant manner.

As used herein, the term “subject” is intended to include human and non-human animals. Preferred subjects include human patients in need of enhancement of an immune response. The methods are particularly suitable for treating human patients having a disorder that can be treated by augmenting the T-cell mediated immune response. In a particular embodiment, the methods are particularly suitable for treatment of cancer cells in vivo.

In certain embodiments it is preferred to use the invention in subjects that are not checkpoint inhibitor refractory patients.The term “checkpoint inhibitor refractory patient” refers to a patient identified as non-responsive to checkpoint inhibitor therapy. A refractory patient may exhibit an innate (primary) resistance to checkpoint inhibitor therapy. Innate resistance may be demonstrated by a lack of response or an insufficient response to said checkpoint inhibitor therapy for at least about 8 weeks, or 12 weeks from the first dose. A refractory patient may exhibit an acquired (secondary) resistance to checkpoint inhibitor therapy. Acquired resistance may be demonstrated by an initial response to said checkpoint therapy but with a subsequent relapse and progression of one or more tumours. Checkpoint inhibitor refractory patients can be non-responsive to any checkpoint inhibitor, non-limiting example include CTLA-4, PD-1, PD-L1, PD-L2, B7-H3, B7-H4, B7-H6, A2AR, IDO, TIM-3, BTLA, VISTA, LAG-3 inhibitors, or combinations thereof.

As used herein, the terms “concurrent administration” or “concurrently” or “simultaneous” mean that administration occurs on the same day. The terms “sequential administration” or “sequentially” or “separate” mean that administration occurs on different days.

“Simultaneous” administration, as defined herein, includes the administration of the one or more immunomodulators within about 2 hours or about 1 hour or less of each other, even more preferably at the same time.

“Separate” administration, as defined herein, includes the administration of the one or more immunomodulators, more than about 12 hours, or about 8 hours, or about 6 hours or about 4 hours or about 2 hours apart.

“Sequential” administration, as defined herein, includes the administration of the one or more immunomodulators in multiple aliquots and/or doses and/or on separate occasions. The immunomodulators may be administered to the patient after before and/or after administration of the other. Alternatively, the immunomodulator is continued to be applied to the patient after treatment with the other.

The use of the alternative (e.g., “or”) should be understood to mean either one, both, or any combination thereof of the alternatives. As used herein, the indefinite articles “a” or “an” should be understood to refer to “one or more” of any recited or enumerated component.

As used herein, “about” means within an acceptable error range for the particular value as determined by one of ordinary skill in the art, which will depend in part on how the value is measured or determined, i.e., the limitations of the measurement system. For example, “about” can mean within 1 or more than 1 standard deviation per the practice in the art. Alternatively, “about” can mean a range of up to 20%. When particular values are provided in the application and claims, unless otherwise stated, the meaning of “about” should be assumed to be within an acceptable error range for that particular value.

The microneedle device and/or patch according to the invention, may be constructed from a variety of materials, including metals, ceramics, semiconductors, organics, polymers, etc., as well as composites thereof. By way of example, pharmaceutical grade stainless steel, titanium, nickel, iron, gold, tin, chromium, copper, alloys of these or other metals, silicon, silicon dioxide, and polymers may be utilized. Typically, the device is formed of a biocompatible material that is capable of carrying a pattern of structures as described herein on a surface. The term “biocompatible” generally refers to a material that does not substantially adversely affect the cells or tissues in the area where the device is to be delivered. It is also intended that the material does not cause any substantially medically undesirable effect in any other areas of the living subject. Biocompatible materials may be synthetic or natural. Some examples of suitable biocompatible materials, which are also biodegradable, include polymers of hydroxy acids such as lactic acid and glycolic acid polylactide, polyglycolide, polylactide-co-glycolide, copolymers with polyethylene glycol, polyanhydrides, poly(ortho)esters, polyurethanes, poly(butyric acid), poly(valeric acid), and poly(lactide-co-caprolactone). Other suitable materials may include, without limitation, polycarbonate, polymethacrylic acid, ethylenevinyl acetate, polytetrafluorethylene, and polyesters. The device may likewise be non-porous or porous in nature, may be homogeneous or heterogeneous across the device with regard to materials, geometry, solidity, and so forth, and may have a rigid fixed or a semi-fixed shape.

The device according to the invention may include an array of individual needles, each formed to a size and shape so as to penetrate the stratum corneum without breakage of the individual microneedles. Microneedles may be solid, porous, or may include a hollow portion. A microneedle may include a hollow portion, e.g., an annular bore that may extend throughout all or a portion of the needle, extending parallel to the direction of the needle or branching or exiting at a side of the needle, as appropriate.

In a further embodiment, the microneedles or, immunomodulator according to the invention, comprises biodegradable sustained release compositions comprising a polymeric matrix selected from the group consisting of carboxy methyl cellulose (CMC) polymer, polylactide (PLA), polyglycolide (PGA), polybutylene succinate (PBS), polyhydroxyalkanoate (PHA), polycaprolactone acid lactone (PCL), polyhydroxybutyrate (PHB), polyvinyl pyrrolidone (PVP), polyvinyl alcohol (PVA), cyclic-olefin copolymer (COC), PHB and PHV copolymer (PHBV), poly lactic acid (PLA)-polyethylene glycol (PEG) copolymers (PLEG), sodium hyaluronate, and combinations thereof.

Examples of other non-limiting polymeric matrix materials that may be used include silicone, hydrogels such as crosslinked poly(vinyl alcohol) and poly(hydroxy ethylmethacrylate), acyl substituted cellulose acetates and alkyl derivatives thereof, partially and completely hydrolyzed alkylene-vinyl acetate copolymers, unplasticized polyvinyl chloride, crosslinked homo- and copolymers of polyvinyl acetate, crosslinked polyesters of acrylic acid and/or methacrylic acid, polyvinyl alkyl ethers, polyvinyl fluoride, polycarbonate, polyurethane, polyamide, polysulphones, styrene acrylonitrile copolymers, crosslinked poly(ethylene oxide), poly(alkylenes), poly(vinyl imidazole), poly(esters), poly(ethylene terephthalate), polyphosphazenes, and chlorosulphonated polyolefines, and combinations thereof Other non-limiting examples include polymers made of dextran sulfate, galacturonic acid, alginates, mannuronic acid, guluronic acid, sodium hyaluronate, chondroitin sulfates, heparin, chitin, chitosan, glycosaminoglycans, proteoglycans, and combinations thereof.

Biodegradable compositions of the invention may typically be formulated with a Mycobacterium loading of about 0.1% to about 95% by weight of the total composition. For example, the sustained release composition may contain a polymer matrix and about 2% to about 20%, about 2% to about 30%, about 2% to about 40%, about 2% to about 50%, about 2% to about 60%, about 2% to about 70%, about 2% to about 80%, or about 2% to about 85% of Mycobacterium by weight of the total composition.

The biodegradable compositions may optionally exhibit sustained release over hours, days or weeks, or in some embodiments, may release the Mycobacterium immediately and rapidly after implant or injection (immediate release), and continues thereafter for a period of time (sustained release). The immediate release period may be, for example, from 1 minute to 5 minutes, from 1 minute to 10 minutes, from 1 minute to 30 minutes, from 1 minute to 60 minutes, from 1 minute to 3 hours, from 1 minute to 6 hours, or from 1 minute to 24 hours, from 1 minute to 2 days, and from 1 minute to 3 days. In other embodiments, the sustained release period may be for about 2 days to about 4 days, about 2 days to about 7 days, about 1 week to about 2 weeks.

In some embodiments, the microneedle array may be in the form of a patch. The microneedles are biosoluble and/or biodegradable. Thus, the biodegradable microneedle dissolves in the skin after the patch is applied. In some embodiments, microneedles detach from patch when the patch is applied to a surface and removed, and then the microneedles dissolve. In some embodiments, only the tip of the microneedles dissolve, and the remaining portion of the microneedle is still attached to the basor patch. Having dissolved, the material will then be metabolized to give harmless end-products. The timescale for dissolving after applying the patch can vary, but dissolving will typically commence immediately after applying the patch (e.g. within 10 seconds) and may continue for e.g. up to 1 minute, 5 minutes, 10 minutes, 20 minutes, 30 minutes, 1 hour, 5 hours, 10 hours, or 24 hours, until the microneedle has fully dissolved. Materials with suitable in vivo dissolving kinetics are readily available to the skilled person.

In some embodiments, the biodegradable sustained release compositions may further comprise pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The compositions disclosed herein may further contain a hydrogel. Non-limiting examples of hydrogels include methyl cellulose (MC), ethyl cellulose (EC), ethyl methyl cellulose (EMC), hydroxyethyl cellulose (HEC), hydroxylpropyl cellulose (HPC), hydroxymethyl cellulose (HMC), hydroxypropylmethyl cellulose (HPMC), ethylhydroxyethyl cellulose (EHEC), hydroxyethylmethy cellulose (HEMC), methylhydroxyethyl cellulose (MHEC), methylhydroxypropylcellulose (MHPC), and hydroxyethylcarboxymethyl cellulose (HECMC).

Table 1 below presents various methodologies and formulation approaches for fabricating solid microneedles according to the invention.

| Methods | Materials | Dimensions (μm) |
|---|---|---|
| Separable dissolving Arrow | (polydimethylsiloxane (PDMS) | 600 μm-long PVP/PVA |
| Heads | Sylgard, | arrowhead capped onto |
| | Metal shaft | a metal shaft with an |
| | Water soluble excipients-PVP, | exposed length of 600 |
| | sucrose | μm and a 100 μm |
| | PLGA | overlap. |
| Dissolving | Mixture Inulin, | basal diameter: |
| | water, dextrin | 3.24 ± 0.16 and 0.55 ± 0.03 |
| | | mm |
| Pyramidal dissolving Polymer | polyvinyl alcohol (PVA), | base width × base |
| | Polyvinylpyrrolid one (PVP) | depth × needle height: |
| | A = Pyramidal MN | 300 μm × 300 μm × 600 |
| | B = Extended pyramidal MNs | μm. |
| | C = Pedestal MNs | 300 μm × 300 μm × 900 |
| | | μm. |
| | | 340 μm × 340 μm × 900 |
| | | μm) |
| | | 10 × 10 array |
| Deep reactive Ion etching | Chromium | 120 μm length, <1 μm tip |
| | Silicon wafers | diameter |
| Dissolving. Fabricated by a | Maltose | 1200 μm length |
| drawing technique to create a | | 60 μm tip diameter |
| sharp tip | | |
| Cutting metal using infrared | Metal | 1000 μm length, |
| laser, manually bending the MN | | 50 μm × 200 μm cross |
| structure, electropolished | | section at base, tapered |
| | | to a sharp tip (angle 20°), |
| | | 106 array |
| Cutting metal using infrared | Metal | 1000 μm length, |
| laser, manually bending the MN | | 50 μm × 200 μm cross |
| structure, electropolished | | section at base, tapered |
| | | to a sharp tip (angle 20°), |
| | | 106 array |
| Silicon master mould to make | Sugar glass MN: | 200 μm base |
| PDMS inverse mould | Trehalose/mannitol (50:50 w/w) | 20 μm tip |
| | Trehalose dehydrate/sucrose | 300 μm height |
| | (75:25 w/w) | |
| | Trehalose/sucrose (75:25 w/w) | |
| | Trehalose/sucrose (50:50 w/w) | |
| Stainless steel MN | Stainless steel | 750 pm height |
| produced by chemical | | 200 pm × 50 pm at the |
| etching | | base. Single row of 5 |
| | | MNs |
| Dissolving MN | Starch/gelatin (1:1 ratio) | 600 pm |
| | PDMS mould | height 300 |
| | | pm base 5 |
| | | pm tip |

Solid microneedles according to the invention, are suitably fabricated by a method selected from dry-etching, wet etching, 3D-printing, laser ablation, micromolding, magnetorheological drawing, lithography or electroplating.

Coated microneedles according to the invention, are suitably fabricated by a method selected from dry-etching, wet etching, 3D-printing, laser ablation, micromolding, magneto rheological drawing, lithography or electroplating.

Dissolving microneedles according to the invention, are suitably fabricated by a method selected from micromolding, ultrasonic welding, 3D-printing, micromachining or ion etching.

Hollow microneedles according to the invention, are suitably fabricated by a method selected from lithographic molding, X-ray photolithography, Microelectromechanical systems (MEMS) or 3D-printing.

Table 2 below presents a selection of microneedle device technologies for use according to the invention, said patents and patent application herein incorporated by reference.

| Patent Number | Date of Filing | Applicant |
|---|---|---|
| US 2010042050 | 16^{th} Apr. 2007 | Nemaura Pharma Ltd, USA |
| WO 2011016230 | 4^{th} Aug. 2010 | Medrx Co., Ltd., Japan |
| WO 2011084951, | 4^{th} Jan. 2011, | Ratio, Inc., |
| US 20110172645 | 8^{th} Jan. 2010 | USA |
| WO 2011014514 | 27^{th} Jul. 2010 | 3M Innovative Properties |
| CA 2696810 | 15^{th} Jan. 2010 | Bioserentach |
| JP 2011078618 | | Co. Ltd., |
| WO 2011043086 | | Japan |
| AU 2010201434 | | |
| KR 2011067009 | | |
| EP 2343102 A1 | | |
| WO 2013096026 | 12^{th} Dec. 2012 | 3M Innovative Properties |
| WO 2013066262 | 2^{nd} Nov. 2011 | Singapore |
| EP 2289843 | 31^{st} Aug. 2009 | University College Cork- National |
| | | University of Ireland, Cork, |
| | | Ireland. |
| JP 2011083387 | 14^{th} Oct. 2009 | Kyushu Institute of Technology, |
| | | Japan; Nichiba Co., Ltd. |
| CN 102000020 | 17^{th} Nov. 2010 | Beijing Pharmaceutical Research |
| | | Institute, Henan Lingrui |
| | | Pharmaceutical Co., Ltd., |
| CN 103263727 | 22^{nd} May 2013 | Tsinghua University, People |
| | | Republic of China. |
| US 20130030374 | 11 Oct. 2012 | Toppan Printing Co., Ltd. |
| CN 103181887 | 30 Dec. 2011 | Shanghai No. 7 People's Hospital, |
| | | People Republic of China |
| GB2472778A | 17^{th} Aug. 2009 | PANGAEA LAB LTD |
| WO 2011026144 or | 31^{st} Aug. 2009 | AllTranz Inc., USA |
| 20110052694 | | |
| US 20110118560, | 13^{th} Nov. 2009, | Searete LLC, USA |
| 20110118656, US | 19^{th} Feb. 2010, | |
| 20110117150, US | 9^{th} Mar. 2010 | |
| 20110118652, US | | |
| CN 101991846 | 17^{th} Nov. 2010 | Chifeng Boen Pharmaceutical Co., |
| | | Ltd., People Republic of China |
| US 20130171722 | 3^{rd} Jan. 2012 | City University of Hong Kong, |
| | | Hong Kong |

Other preferred microneedle devices for use according to the invention include: North Carolina State University (as described in WO2017/151727), Debioject microneedle (Debiotech, Switzerland), Micronject600 (NaoPass, Israel, as described in WO2008/047359), Nanopatch (Vaxxas, USA), SOFUSA (Kimberly-Clark/, Sorrento Therapeutics Inc., USA, as described in WO2017/189259 and WO2017/189258), Micron Biomedical's dissolving microarray, and the MIMIX dissolving, controlled release microarray (Vaxess, USA), plus 3M's Microstructured Transdermal Systems (MTS) or Zosano's titanium microprojection array employed in Qytpta (zolmitriptan intracutaneous microneedle system) or Macroflux.

Additional examples of suitable microneedle devices include: the SCS Microinjector® from Clearside Biomedical, which is composed of a syringe and two 30-gauge hollow microneedles of varying lengths, each less than 1.2 millimetres; the Microinfusor® developed by Becton Dickinson (BD) Technologies, which is a hollow microneedle system that allows delivery of a wide range of drugs to the subcutaneous tissue over a period of time; Corium's MicroCar® system, which consists of dissolving microneedles; Mercator MedSystems, Inc. device named Bullfrog® Micro-Infusion Device which may safely inject therapeutic molecules through blood vessel walls into adventitial tissues, where the device is tipped with a balloon-sheathed microneedle.

Another suitable hollow microneedle system is the AdminPen from AdminMed nanoBioSciences LLC, such as the AdminPen 600 (which has 187×500 um-tall microneedles on a 1 cm² circular microneedle array. The microneedle array is made of SS 316L stainless steel), or the AdminPen 700 (127×700 um-tall microneedles).

Alternative dissolving microneedle patches include DrugMAT and VaxMAT.

Preferable microneedle devices for use according to the invention are those which have received 510(k) marketing clearance from the FDA and/or are CE Marked and/or are certified Class II Medical Devices.

In an embodiment, the present invention provides an immunomodulator for use in the treatment, reduction, inhibition or control of cancer in a subject, wherein the immunomodulator comprises a whole cell, non-viable Mycobacterium and wherein said immunomodulator is to be administered into the skin of said subject via a microneedle device comprising a plurality of microneedles.

In an embodiment, the microneedles are hollow. In a separate embodiment the microneedles are solid or porous.

In a further embodiment, the plurality of microneedles are deployed in a line, square, circle, grid or array.

In a further embodiment, the microneedle device includes between 2 and 5000 microneedles, such as between 2 and 500 microneedles, and deployed on said device as between 2 and 2000 microneedles per square centimetre, such as between 4 and 1500 microneedles per square centimetre, or between 10 and 1000 microneedles per square centimetre.

In a further embodiment, any or all of the microneedles are between 2 and 2000 microns in length, such as between 20 and 1000 microns, or between 50 and 500 microns, or between 100 and 400 microns.

In a further embodiment, any or all of the microneedles are configured to deliver the immunomodulator intradermally, subcutaneously or subdermally, optionally wherein said immunomodulator is delivered to the lymphatic vessels.

In a further embodiment, the said immunomodulator is coated onto or embedded within at least a portion of any or all of the microneedles, optionally wherein the microneedles are implanted into or removable from the skin. Preferably, said coating or microneedle is dissolvable. or hydratable upon contact with the skin in order to provide immediate or sustained release of said immunomodulator.

In a further embodiment, wherein said microneedles are hollow and said immunomodulator is delivered intradermally, subcutaneously or subdermally as a suspension through said microneedles via one or more apertures situated thereon, optionally wherein said microneedles are implanted into or removable from the skin.

In a further embodiment, said one or more checkpoint inhibitor is an antibody selected from ipilimumab, nivolumab, pembrolizumab, atezolizumab, tremelimumab, and combinations thereof. Suitably, said checkpoint inhibitor is pembrolizumab, preferably wherein said subject has mismatch-repair deficient tumours and/or exhibits PD-L1 expression in at least 10%, or 20%, or 30%, or 40%, or 50% or more of tumour cells, as measured using the SP142 immunohistochemistry antibody assay.

In a further embodiment, said one or more checkpoint inhibitors are selected from ipilimumab, nivolumab, pembrolizumab, atezolizumab, durvalumab, tremelimumab, spartalizumab, avelumab, sintilimab, toripalimab, MGA012, MGD013, MGD019, enoblituzumab, MGD009, MGC018, MED10680, PDR001 , FAZ053, TSR022, MBG453, relatlimab (BMS986016), LAG525, IMP321, REGN2810 (cemiplimab), REGN3767, pexidartinib, LY3022855, FPA008, BLZ945, GDC0919, epacadostat, indoximod, BMS986205, CPI-444, MED19447, PBF509, lirilumab and combinations thereof.

Suitable combinations of said checkpoint inhibitors according to the invention include: durvalumab+tremelimumab, nivolumab+ipilimumab, pembrolizumab+ipilimumab, MED10680+durvalumab, PDR001+FAZ053, Nivolumab+TSR022, PDR001+MBG453, Nivolumab+BMS 986016 (relatlimab), PDR001+LAG 525, Pembrolizumab+IMP321, REGN2810 (cemiplimab)+REGN3767.

The invention may further include co-stimulatory checkpoint therapy which comprises administration of one or more checkpoint inhibitors, selected from a cell, protein, peptide, antibody, ADC (antibody-drug conjugate), Fab fragment (Fab), F(ab')2 fragment, diabody, triabody, tetrabody, probody, single-chain variable region fragment (scFv), disulfide-stabilized variable region fragment (dsFv), or other antigen binding fragment thereof, directed against CD27, CD28, CD40, CD122, CD137, 0X40, GITR, ICOS and combinations thereof. Suitable such co-stimulatory checkpoint inhibitors are selected from utomilumab, urelumab, MOXR0916, PF04518600, MEDI0562, GSK3174988, MEDI6469, R07009789, CP870893, BMS986156, GWN323, JTX-2011, varlilumab, MK-4166, NKTR-214 and combinations thereof.

In other embodiments of the invention, the biologically-active agent is thymosin or a purified fraction thereof, such as thymosin alpha or thymalfasin (Zadaxin), optionally injected once per week or delivered nasally or via oral aerosol inhalation. The thymosin or thymalfasin may be administered at the same time and/or via the same route as the Mycobacterium, or at separate times and/or via separate routes of administration.

The Mycobacterium is non-viable, e.g. is a heat-killed Mycobacterium, suitably a whole cell Mycobacterium. Examples of mycobacterial species for use in the present invention include M. vaccae, M. thermoresistibile, M. flavescens, M. duvalii, M. phlei, M. obuense, M. parafortuitum, M. sphagni, M. aichiense, M. rhodesiae, M. neoaurum, M. chubuense, M. tokaiense, M. komossense, M. aurum, M. w, M. tuberculosis, M. microti; M. africanum; M. kansasii, M. marinum; M. simiae; M. gastri; M. nonchromogenicum; M. terrae; M. triviale; M. gordonae; M. scrofu/aceum; M. paraffinicum; M. intracellulare; M. avium; M. xenopi; M. ulcerans; M. diernhoferi, M. smegmatis; M. thamnopheos; M. flavescens; M. fortuitum; M. peregrinum; M. chelonei; M. paratuberculosis; M. leprae; M. lepraemurium and combinations thereof.

Preferably, the non-viable Mycobacterium is non-pathogenic. The non-pathogenic heat-killed Mycobacterium is preferably selected from M. vaccae, M. obuense, M. parafortuitum, M. aurum, M. indicus pranii, M. phlei and combinations thereof.

Preferably, the non-pathogenic non-viable Mycobacterium is selected from M. vaccae, including the strain deposited under accession number NCTC 11659 and associated designations such as SRL172, SRP299, IMM-201, DAR-901, and the strain as deposited under ATCC 95051 (Vaccae™), M. obuense, M. paragordonae (strain 49061), M. parafortuitum, M. aurum, M. phlei, M. indicus pranii, M.w, M. kyogaense (as deposited under DSM 107316/CECT 9546), M. tuberculosis Aoyama B or H37Rv, and combinations thereof, preferably the strain of Mycobacterium obuense deposited under the Budapest Treaty under accession number NCTC 13365.

Methods to enforce such non-viability may include heat-killing, irradiation by gamma waves or electron beam, or subjecting the bacteria to chemicals such as formaldehyde. Such preparation during manufacture would mean the organism is not associated with side-effects known from delivering live or attenuated organisms.

The amount of Mycobacterium administered to the patient in the present invention would be sufficient to elicit a protective immune response in the patient such that the patient's immune system would be able to mount an effective immune response against the tumour, cancer, neoplasia or metastases.

In certain embodiments of the invention, there is provided a containment means comprising the effective amount of non-viable Mycobacterium for use in the present invention, which typically may be from 10³ to 10¹¹ organisms, preferably from 10⁴ to 10¹⁰ organisms, more preferably from 10⁶ to 10¹⁰ organisms, and even more preferably from 10⁶ to 10⁹ organisms. The effective amount of non-viable Mycobacterium for use in the present invention may be from 10³ to 10¹¹ organisms, preferably from 10⁴ to 10¹⁰ organisms, more preferably from 10⁶ to 10¹⁰ organisms, and even more preferably from 10⁶ to 10⁹ organisms. Most preferably the amount of non-viable Mycobacterium for use in the present invention is from 10⁷ to 10⁹ cells or organisms. Typically, the composition according to the present invention may be administered at a dose of from 10⁸ to 10⁹ cells for human and animal use. Alternatively, the dose is from 0.01 mg to 1 mg or from 0.1 mg to 1 mg organisms presented as either a suspension or dry preparation, optionally at a delivery rate of between 0.0001 mg per hour to 10 mg per hour.

In certain embodiments of the invention, the amount of non-pathogenic non-viable Mycobacterium administered is suitably a therapeutically effective amount such as from 10⁴ to 10⁹ cells per unit dose.

In certain embodiments of the invention, the amount of non-pathogenic non-viable Mycobacterium administered is between 0.0001 mg and 1 mg per unit dose, optionally wherein the unit dose is administered on two or more separate occasions.

In other embodiments of the invention, the amount of non-pathogenic non-viable Mycobacterium administered is between 0.001 mg and 1 mg per unit dose, optionally wherein the unit dose is administered on two or more separate occasions.

In certain embodiments of the invention, the subject is administered a priming or initial dose of preferably at least about 0.5 mg per unit dose, and one or more subsequent or boosting doses of not more than about 0.5 mg per unit dose.

In further embodiments of the invention, the amount of non-pathogenic non-viable Mycobacterium administered is between 0.01 mg and 1 mg per unit dose, optionally wherein the unit dose is administered on two or more separate occasions separated by at least 7 days or more, such as administration on each of day 0, day 14 (+/−1, 3 or 5 days or more), and optionally day 30 (+/−5, 7 or 10 days or more) or day 45 (+/−7, 10 or 14 days or more).

In some embodiments, the amount of non-pathogenic non-viable Mycobacterium administered may be from 0.0001 mg to 1 mg per dose wherein the dose is administered 1, 2, 3, 4, 5, 6, 10 or 20 or more times over a number of days, weeks, or months, suitably wherein the Mycobacterium is M. obuense.

In other embodiments of the invention, the amount of non-pathogenic non-viable Mycobacterium administered may be from 0.0001 mg to 1 mg per dose, wherein the dose initially comprises two injections of 0.5 mg in each deltoid, or two injections of 1.0 mg in each deltoid, followed by a second dose of either 0.5 or 1.0 mg 7 or 14 days or more later, optionally with further doses administered over the subsequent weeks or months.

Alternatively, the amount of non-pathogenic non-viable Mycobacterium administered may be from 0.0001 mg to 1 mg per dose, administered three times per day for three consecutive days,

M. vaccae and M. obuense are particularly preferred. M. obuense is sometimes referred to as IMM-101 and a suitable exemplary strain is deposited under the NCTC accession number, NCTC13365. A suitable exemplary strain of M. vaccae is deposited under the NCTC accession number NCTC 11659.

In a further embodiment of the invention, the non-pathogenic, non-viable Mycobacterium is the rough variant and/or whole cell.

In preferred embodiments of the invention, the non-pathogenic, non-viable Mycobacterium obuense is the rough variant.

In preferred embodiments of the invention, the non-pathogenic, non-viable Mycobacterium obuense is in a substantially whole cell form, such as where more than 50% or more of the mycobacteria in suspension are greater than 1 micron in diameter, as measured by laser diffraction, or is in a form which has not been exposed to high pressure processing or other conditions to induce cell lysis.

As would be understood by the skilled person, rough variants of M. obuense would lack cell surface-associated glycopeptidolipids (GPL) resulting in a characterised rough morphology with non-motile and non-biofilm-forming properties, as described in Roux et al. 2016, Open Biol 6: 160185.

In a further embodiment of the invention, the non-pathogenic, non-viable Mycobacterium is presented as a lysate, homogenate or sonicate of a whole cell, including fractions thereof.

M. vaccae and M. obuense induce a complex immune response in the host. Treatment with these preparations will stimulate innate and type-1 immunity, including Th1 and macrophage activation and cytotoxic cell activity. They also independently down-regulate inappropriate Th2 responses via immunoregulatory mechanisms. This restores the healthy balance of the immune system.

The demonstrated mechanism of action (MoA) of M. obuense is a multi-targeted, systemic immunomodulation of the innate and adaptive immune system, including but not limited to a rapid type 1 immune response with, systemic expansion of IFNγ producing activated NK cells, NKT-cells and γδ T-cells, as well as CD4+ (Th1) and CD8+ (perforin and granzyme producing CTL) T-cells. IMM-101 (M. obuense) activated Vδ2 T-cells show enhanced effector responses, upregulated granzyme B expression, enhanced production of IFN-γ and TNF-α, and enhanced degranulation in response to tumour cells (Fowler et al. 2012 Cancer Immunol I mmunother 61: 535-547).

It has been shown in experiments with mouse and human immune cells that IMM-101 (a non-viable, whole cell M. obuense preparation in clinical trials) is a strong activator of antigen presenting macrophages and dendritic cells (DCs) and that the DC activation leads to a typical Type 1 immune response, with formation and activation of CD4+ T-helper 1 lymphocytes (Th1s) and CD8+ cytotoxic T lymphocytes (CTLs) and increased production of the cytokine interferon-y (IFN-g) in the lymph nodes in which IMM-101 activated DCs are present. In addition, other experiments have shown that IMM-101 also increases the number and activation of natural killer cells (NKs) and T cells expressing gamma/delta receptors (gd-T cells). This and CTLs require tumour cells to express specific tumour-associated antigens (TAAs) for their attack, whereas NK and gd-T cells do not require the presence of such TAAs to kill tumour cells. These four different immune cells work in concert to form an effective anti-tumour response. In relation to cancer, it is likely that the formation of IFN-y producing CTLs is the most important result from IMM-101 treatment, since the observed anti-tumour effect of IMM-101 could be completely abrogated by the depletion of CD8+ T cells in a pancreas cancer model.

IMM-101's ability to activate macrophages may not only assist in the activation of

DCs through the release of pro-inflammatory macrophage-derived cytokines (such as IL-12 required for skewing DCs into Type 1 immune responses), but may also be of importance for changing tumour associated immunosuppressive type 2 macrophages into tumour aggressive type 1 macrophages. This latter feature was shown for a similar heat-killed Mycobacterium, M. indicus pranii.

An important feature of IMM-101 is its ability to activate and mature DCs into a sub-class of dendritic cells known as cDC1s (i.e. DCs that are required for Type 1 immune responses). It has been shown that activation of sufficient numbers of cDC1 s is a prerequisite for CPIs to be effective.

It is generally believed that a Type 1 immune response resulting in INF-gamma producing Th-1 cells and CTLs, which specifically attack TAA-expressing tumour cells, and activated NK and gd-T cells, which attack tumours through other mechanisms, is the body's main mechanism and a pre-requisite for an effective anti-cancer response and should therefore be at the core of any immune-mediated cancer treatment—preclinical data show that IMM-101 is capable of stimulating such required Type 1 immune responses.

The impact of IMM-101 on DC priming has been studied in vitro and it was found that IMM-101 displayed a dose-dependent ability to induce phenotypic activation and cytokine production for both human and murine DCs. For example, GM-CSF derived murine DC displayed a dose dependent response to IMM-101, with elevated membrane expression of CD80, CD86, CD40 and MHC II and increased production of IL-6, IL-12p40 and nitric oxide, which are all molecules that are essential for effective antigen-dependent activation of T cells. Moreover, human monocyte-derived DCs showed a similar response to IMM-101, with up-regulation of CD80, CD86 and MHC II and secretion of a number of relevant cytokines, showing clear activation of DCs. Exposure to IMM-101 in vitro also showed that IMM-101 functionally affects the DCs by enhancing their ability to process and present antigen.

In vivo experiments have shown that IMM-101 activated DCs are able to activate CD8+ and CD4+ T cells and promote secretion of IFN-g following re-stimulation of draining lymph node cell preparations, 7 days after subcutaneous adoptive transfer of IMM-101 (in vitro) activated GM-CSF derived murine DCs into naive recipient mice.

IMM-101 (Mycobacterium obuense) in particular, has been shown to be well tolerated in over 300 patients with advanced cancer in clinical trials and compassionate use programs. IMM-101 demonstrated further clinical benefit in a randomized Phase 2 study in advanced pancreatic cancer (Dalgeish et al. 2016 BR J Cancer 115(7): 789 796).

The present invention may be used to treat a neoplastic disease, such as solid or non-solid cancers. As used herein, “treatment” encompasses the prevention, reduction, control and/or inhibition of a neoplastic disease. Such diseases include a sarcoma, carcinoma, adenocarcinoma, melanoma, myeloma, blastoma, glioma, lymphoma or leukemia. Exemplary cancers include, for example, carcinoma, sarcoma, adenocarcinoma, melanoma, neural (blastoma, glioma, or glioblastomas), mesothelioma and reticuloendothelial, lymphatic or haematopoietic neoplastic disorders (e.g., myeloma, lymphoma or leukemia). In particular aspects, a neoplasm, tumour or cancer includes a lung adenocarcinoma, lung carcinoma, diffuse or interstitial gastric carcinoma, colon adenocarcinoma, prostate adenocarcinoma, esophagus carcinoma, gastro-oesophageal carcinoma, breast carcinoma, pancreas adenocarcinoma, ovarian adenocarcinoma, adenocarcinoma of the adrenal gland, adenocarcinoma of the endometrium or uterine adenocarcinoma.

Neoplasia, tumours and cancers include benign, malignant, metastatic and non-metastatic types, and include any stage (I, II, III, IV or V) or grade (G1, G2, G3, etc.) of neoplasia, tumour, or cancer, or a neoplasia, tumour, cancer or metastasis that is progressing, worsening, stabilized or in remission. Cancers that may be treated according to the invention include but are not limited to cells or neoplasms of the bladder, blood, bone, bone marrow, brain, breast, colon, esophagus, gastrointestines, gum, head, kidney, liver, lung, nasopharynx, neck, ovary, prostate, skin, stomach, testis, tongue, or uterus. In addition, the cancer may specifically be of the following histological type, though it is not limited to the following: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumour, malignant; bronchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma with squamous metaplasia; thymoma, malignant; ovarian stromal tumour, malignant; thecoma, malignant; granulosa cell tumour, malignant; androblastoma, malignant; Sertoli cell carcinoma; Leydig cell tumour, malignant; lipid cell tumour, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumour; Mullerian mixed tumour; nephroblastoma; hepatoblastoma; carcinosarcoma; mesenchymoma, malignant; Brenner tumour, malignant; phyllodes tumour, malignant; synovial sarcoma; mesothelioma, malignant; dysgerminoma; embryonal carcinoma; teratoma, malignant; struma ovarii, malignant; choriocarcinoma; mesonephroma, malignant;

hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumour of bone; Ewing's sarcoma; odontogenic tumour, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumour; meningioma, malignant; neurofibrosarcoma; neurilemmoma, malignant; granular cell tumour, malignant; malignant lymphoma; Hodgkin's disease; Hodgkin's; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. Preferably, the neoplastic disease may be tumours associated with a cancer selected from prostate cancer, liver cancer, renal cancer, lung cancer, breast cancer, colorectal cancer, pancreatic cancer, brain cancer, hepatocellular cancer, lymphoma, leukaemia, gastric cancer, cervical cancer, ovarian cancer, thyroid cancer, melanoma, head and neck cancer, skin cancer and soft tissue sarcoma and/or other forms of carcinoma. The tumour may be metastatic or a malignant tumour.

More preferably, the neoplastic disease to be treated is pancreatic cancer, breast cancer, lung cancer, ovarian cancer, gastro-oesophageal cancer, glioblastomas, colorectal cancer, prostate cancer and skin cancer and other solid tumours. In a preferred embodiment the neoplastic disease to be treated is pancreatic cancer. In a further preferred embodiment, the neoplastic disease to be treated is lung cancer. In a further preferred embodiment, the neoplastic disease to be treated is colorectal cancer.

More preferably, the cancer is selected from pancreatic, colorectal, prostate, gastro-oesophageal, skin, breast, brain, glioblastoma, melanoma, sarcoma or ovarian cancer, optionally wherein said cancer is locally advanced, inoperable, borderline operable, or resectable.

In an embodiment of the invention, the immunomodulator is used to reduce or inhibit metastasis of a primary tumour or cancer to other sites, or the formation or establishment of metastatic tumours or cancers at other sites distal from the primary tumour or cancer, thereby inhibiting or reducing tumour or cancer relapse or tumour or cancer progression.

In an embodiment of the invention, said cancer is preferably metastatic, disseminated or micrometastatic, such as metastatic pancreatic ductal adenocarcinoma (mPDAC).

In further embodiments, methods of the invention include, one or more of the following: 1) reducing or inhibiting growth, proliferation, mobility or invasiveness of tumour or cancer cells that potentially or do develop metastases, 2) reducing or inhibiting formation or establishment of metastases arising from a primary tumour or cancer to one or more other sites, locations or regions distinct from the primary tumour or cancer; 3) reducing or inhibiting growth or proliferation of a metastasis at one or more other sites, locations or regions distinct from the primary tumour or cancer after a metastasis has formed or has been established, 4) reducing or inhibiting formation or establishment of additional metastasis after the metastasis has been formed or established, 5) prolonged overall survival, 6) prolonged progression free survival, or 7) disease stabilisation.

The therapeutic effect may not take effect immediately. For example, treatment may be followed by an increase in the neoplasia, tumour or cancer cell numbers or mass, but over time eventual stabilization or reduction in tumour cell mass, size or numbers of cells in a given subject may subsequently occur.

Additional adverse symptoms and complications associated with neoplasia, tumour, cancer and metastasis that can be inhibited, reduced, decreased, delayed or prevented include, for example, nausea, lack of appetite, lethargy, pain and discomfort. Thus, a partial or complete decrease or reduction in the severity, duration or frequency of an adverse symptom or complication associated with or caused by a cellular hyperproliferative disorder, an improvement in the subject's quality of life and/or well-being, such as increased energy, appetite, psychological well-being, are all particular non-limiting examples of therapeutic benefit.

A therapeutic benefit or improvement therefore can also include a subjective improvement in the quality of life of a treated subject. In an additional embodiment, a method prolongs or extends lifespan (survival) of the subject. In a further embodiment, a method improves the quality of life of the subject.

In an embodiment of the invention, the checkpoint inhibitor therapy comprising administration of a blocking agent, selected from a cell, protein, peptide, antibody or antigen binding fragment thereof, directed against CTLA-4, PD-1, PD-L1, PD-L2, LAG-3, B7-H3, B7-H4, B7-H6, A2AR, or IDO, and combinations thereof, in combination with a whole cell, non-viable Mycobacterium, is used to reduce or inhibit metastasis of a primary tumour or cancer to other sites, or the formation or establishment of metastatic tumours or cancers at other sites distal from the primary tumour or cancer thereby inhibiting or reducing tumour or cancer relapse or tumour or cancer progression. Preferably, the blocking agent is directed against CTLA-4, PD-1, or PD-L1, and combinations thereof. Most preferably, the blocking agent is directed against PD-L1.

In a separate embodiment, said checkpoint inhibitor is ipilimumab, preferably wherein said ipilimumab is administered at a dose of up to about 0.3, 0.5 or 2 mg/kg or 3 mg/kg 5 mg/kg or 10 mg/kg or less every three weeks, optionally for a maximum of four administrations, this may then followed by a second phase in the treatment of melanoma in which nivolumab monotherapy is administered intravenously at either 240 mg every 2 weeks or at 480 mg every 4 weeks, For the monotherapy phase, the first dose of nivolumab should be administered; 3 weeks after the last dose of the combination of nivolumab and ipilimumab if using 240 mg every 2 weeks; or, 6 weeks after the last dose of the combination of nivolumab and ipilimumab if using 480 mg every 4 weeks.

In a separate embodiment, said checkpoint inhibitor is nivolumab, preferably wherein said nivolumab is administered at a dose of at least 0.1, 1, 3, or 5 or 10 mg/kg or more every four weeks, optionally wherein said subject exhibits PD-L1 expression in at least 1%, 5% or 10% or more of tumour cells, as measured using the SP142 immunohistochemistry antibody assay.

In a separate embodiment, said checkpoint inhibitor is atezolizumab, preferably wherein said atezolizumab is administered at a dose of at least 0.1, 1, 3, 5, 7, 10 or 15 mg/kg or more every three weeks, optionally wherein said subject exhibits PD-L1 expression in at least 1%, 5% or 10% or more of tumour cells and/or tumour-infiltrating immune cells selected from B-cells and NK cells, as measured using the SP142, 5H1, 28-8, 22C3, and SP263 immunohistochemistry antibody assay from Ventana or Dako.

In a further embodiment of the invention, there is provided a combination therapy for treating cancer, comprising an immunomodulator and checkpoint inhibitor(s), wherein said checkpoint inhibition therapy comprises administration of a blocking agent, selected from a cell, protein, peptide, antibody or antigen binding fragment thereof, directed against CTLA-4, PD-1, PD-L1, PD-L2, LAG-3, B7-H3, B7-H4, B7-H6, A2AR, or IDO, and combinations thereof, wherein the immunomodulator comprises a whole cell, non-viable Mycobacterium, with the potential to elicit potent and durable immune responses with enhanced therapeutic benefit and more manageable toxicity. Preferably, the blocking agent is directed against CTLA-4, PD-1, or PD-L1, and combinations thereof. Most preferably, the blocking agent is directed against PD-L1.

In a further embodiment of the invention, there is provided a combination therapy for treating cancer, comprising an immunomodulator which; (i) stimulates innate and type-1 immunity, including Th1 and macrophage activation and cytotoxic cell activity, and, (ii) independently down-regulates inappropriate Th2 responses via immunoregulatory mechanisms; and, a checkpoint inhibitor, optionally wherein the immunomodulator is a whole cell non-pathogenic, heat-killed Mycobacterium selected from M. vaccae or M. obuense.

In a further embodiment of the invention, the immunomodulator comprises one or more biologically-active agents selected from a therapeutic drug, nutraceutical, vaccine, cell, virus, lysate, vector, gene, mRNA, DNA, nucleic acid, protein, polypeptide, peptide, antibody, bispecific antibody, multi-specific antibody, ADC (antibody-drug conjugate), Fab fragment (Fab), F(ab')2 fragment, diabody, triabody, tetrabody, probody, single-chain variable region fragment (scFv), disulfide-stabilized variable region fragment (dsFv), or other antigen binding fragment thereof, optionally wherein said biologically-active agent is an antigen or antigenic determinant.

In a further embodiment of the invention, the one or more biologically-active agents is a checkpoint inhibitor directed against CTLA-4, PD-1, PD-L1, PD-L2, B7-H3, B7-H4, B7-H6, A2AR, IDO, TIM-3, BTLA, VISTA, TIGIT, LAG-3, CD40, KIR, CEACAM1, GARP, PS, CSF1R, CD94/NKG2A, TDO, TNFR, DcR3 and combinations thereof.

In an embodiment of the invention is provided a method for treating cancer and/or preventing the establishment of metastases by employing a checkpoint inhibitor selected from a cell, protein, peptide, antibody or antigen binding fragment thereof, directed against CTLA-4, PD-1, PD-L1, PD-L2, LAG-3, B7-H3, B7-H4, B7-H6, A2AR, or IDO, and combinations thereof, which act synergistically with a whole cell, non-viable Mycobacterium when administered using a microneedle device including one or more solid, porous or hollow microneedles. Preferably, the checkpoint inhibitor employed is directed against CTLA-4, PD-1, or PD-L1, and combinations thereof. Most preferably, the blocking agent is directed against PD-L1.

In a preferred embodiment of the invention is provided a method of treating, reducing, inhibiting or controlling a neoplasia, tumour or cancer in a subject, wherein said method comprises:
(i) providing a microneedle device comprising a plurality of microneedles,
(ii) causing the microneedles to penetrate through a stratum corneum into the skin of the subject and optionally assume an anchored state in which the microneedles are anchored in the skin, and project from the microneedle device,
(iii) delivering into the skin via the microneedle device, preferably subcutaneously, subdermally or intradermally, a quantity of a non-pathogenic non-viable Mycobacterium selected from M. vaccae, including the strain deposited under accession number NCTC 11659 and associated designations such as SRL172, SRP299, IMM-201, DAR-901, and the strain as deposited under ATCC 95051 (Vaccae™); M. obuense, M. paragordonae (strain 49061), M. parafortuitum, M. aurum, M. phlei, M. indicus pranii, M.w, M. kyogaense (as deposited under DSM 107316/CECT 9546), M. tuberculosis Aoyama B or H37Rv, and combinations thereof, preferably the strain of Mycobacterium obuense deposited under the Budapest Treaty under accession number NCTC 13365.

The subject is preferably not a checkpoint inhibitor refractory patient.

In a preferred embodiment of the invention, said method further comprises simultaneously, separately or sequentially administering to the subject, one or more biologically-active agents selected from a therapeutic drug, nutraceutical, vaccine, cell, virus, lysate, vector, gene, mRNA, DNA, nucleic acid, protein, polypeptide, peptide, antibody, bispecific antibody, multi-specific antibody, ADC (antibody-drug conjugate), Fab fragment (Fab), F(ab')2 fragment, diabody, triabody, tetrabody, probody, single-chain variable region fragment (scFv), disulfide-stabilized variable region fragment (dsFv), or other antigen binding fragment thereof, optionally wherein said biologically-active agent is an antigen or antigenic determinant.

In a further embodiment of the invention, said method further comprises simultaneously, separately or sequentially administering to the subject, one or more biologically-active agents, wherein the one or more biologically-active agents is a checkpoint inhibitor directed against CTLA-4, PD-1, PD-L1, PD-L2, B7-H3, B7-H4, B7-H6, A2AR, IDO, TIM-3, BTLA, VISTA, TIGIT, LAG-3, CD40, KIR, CEACAM1, GARP, PS, CSF1R, CD94/NKG2A, TDO, TNFR, DcR3 and combinations thereof.

In a preferred embodiment of the invention, said method further comprises simultaneously, separately or sequentially administering to the subject, one or more biologically-active agents selected from: thymosin or a purified fraction thereof, such as thymosin alpha or thymalfasin (Zadaxin); an HDAC inhibitor e.g. OKI-179, BRAF inhibitor, MEK inhibitor, EGFR inhibitor, VEGF inhibitor, P13K delta inhibitor, PARP inhibitor, mTOR inhibitor, such as rapamycin, everolimus or metformin; hypomethylating agents, oncolytic virus, TLR agonist including TLR2, 3, 4, 5, 7, 8 or 9 agonists, such as MRx0518 (4D Pharma), STING agonists (including MIW815 and SYNB1891), cancer vaccines such as GVAX or CIMAvax, and optionally, wherein said TLR agonists include mifamurtide (Mepact), Krestin (PSK), MRx0518 (4D Pharma), IMO-2125 (tilsotolimod), CMP-001, MGN-1703 (lefitolimod), entolimod, SD-101, GS-9620, imiquimod, resiquimod, MEDI4736, poly I:C such as rintatolimod (Ampligen), CPG7909, DSP-0509, VTX-2337 (motolimod), MED19197, NKTR-262, G-100 or PF-3512676 and combinations thereof.

In a further embodiment of the invention, said one or more biologically-active agents are administered at the same time and/or via the same microneedle device as the non-pathogenic non-viable Mycobacterium, or at separate times and/or via separate routes of administration.

In a preferred embodiment of the method of the invention, said non-viable Mycobacterium is the rough variant and/or whole cell.

In a further embodiment of the method of the invention, said non-pathogenic, non-viable Mycobacterium is presented as a lysate, homogenate or sonicate of a whole cell, including fractions thereof.

In a further embodiment of the method of the invention, said non-pathogenic, non-viable Mycobacterium is presented as a suspension or in dehydrated form, such as lyophilised or air-dried.

In a further embodiment of the method of the invention, said said neoplasia, tumour or cancer is associated with a cancer selected from prostate cancer, liver cancer, renal cancer, lung cancer, breast cancer, colorectal cancer, breast cancer, pancreatic cancer, brain cancer, glioblastoma, hepatocellular cancer, lymphoma, leukaemia, gastric cancer, cervical cancer, ovarian cancer, thyroid cancer, head and neck cancer, skin cancer including melanoma, or a sarcoma, such as a soft tissue or non-soft tissue sarcoma, preferably wherein said neoplasia, tumour or cancer is associated with a cancer selected from pancreatic, colorectal, prostate, gastro-oesophageal, skin, breast, brain, glioblastoma, melanoma, sarcoma or ovarian cancer, optionally wherein said cancer is locally advanced, inoperable, borderline operable, or resectable.

In a further embodiment of the method of the invention, said neoplasia, tumour or cancer is metastatic, disseminated or micrometastatic, such as metastatic pancreatic ductal adenocarcinoma (mPDAC).

In further embodiments, methods of the invention include, one or more of the following: 1) reducing or inhibiting growth, proliferation, mobility or invasiveness of tumour or cancer cells that potentially or do develop metastases, 2) reducing or inhibiting formation or establishment of metastases arising from a primary tumour or cancer to one or more other sites, locations or regions distinct from the primary tumour or cancer; 3) reducing or inhibiting growth or proliferation of a metastasis at one or more other sites, locations or regions distinct from the primary tumour or cancer after a metastasis has formed or has been established, 4) reducing or inhibiting formation or establishment of additional metastasis after the metastasis has been formed or established, 5) prolonged overall survival, 6) prolonged progression free survival, or 7) disease stabilisation.

In an embodiment of the invention, administration of one or more biologically-active agents selected from a therapeutic drug, nutraceutical, vaccine, cell, virus, lysate, vector, gene, mRNA, DNA, nucleic acid, protein, polypeptide, peptide, antibody, bispecific antibody, multi-specific antibody, ADC (antibody-drug conjugate), Fab fragment (Fab), F(ab')2 fragment, diabody, triabody, tetrabody, probody, single-chain variable region fragment (scFv), disulfide-stabilized variable region fragment (dsFv), or other antigen binding fragment thereof, preferably a checkpoint inhibitor selected from a cell, protein, peptide, antibody or antigen binding fragment thereof, directed against CTLA-4, PD-1, PD-L1, PD-L2, B7-H3, B7-H4, B7-H6, A2AR, IDO, TIM-3, BTLA, VISTA, TIGIT, LAG-3, CD40, KIR, CEACAM1, GARP, PS, CSF1R, CD94/NKG2A, TDO, TNFR, DcR3 and combinations thereof, in combination therapy with a non-pathogenic, non-viable Mycobacterium, provides a detectable or measurable improvement in a condition of a given subject, such as alleviating or ameliorating one or more adverse (physical) symptoms or consequences associated with the presence of a cell proliferative or cellular hyperproliferative disorder, neoplasia, tumour or cancer, or metastasis, i e., a therapeutic benefit or a beneficial effect.

In an embodiment of the invention, said method results in enhanced therapeutic efficacy relative to administration of the one or more biologically-active agents or non-pathogenic, non-viable Mycobacterium alone.

In an embodiment of the invention, said enhanced therapeutic efficacy is measured by increased overall survival time, or by increased progression-free survival.

Preferably, the blocking agent is directed against CTLA-4, PD-1, or PD-L1, and combinations thereof. Most preferably, the blocking agent is directed against PD-1.

A therapeutic benefit or beneficial effect is any objective or subjective, transient, temporary, or long-term improvement in the condition or pathology, or a reduction in onset, severity, duration or frequency of an adverse symptom associated with or caused by cell proliferation or a cellular hyperproliferative disorder such as a neoplasia, tumour or cancer, or metastasis. It may lead to improved survival. A satisfactory clinical endpoint of a treatment method in accordance with the invention is achieved, for example, when there is an incremental or a partial reduction in severity, duration or frequency of one or more associated pathologies, adverse symptoms or complications, or inhibition or reversal of one or more of the physiological, biochemical or cellular manifestations or characteristics of cell proliferation or a cellular hyperproliferative disorder such as a neoplasia, tumour or cancer, or metastasis. A therapeutic benefit or improvement therefore may be, but is not limited to destruction of target proliferating cells (e.g., neoplasia, tumour or cancer, or metastasis) or ablation of one or more, most or all pathologies, adverse symptoms or complications associated with or caused by cell proliferation or the cellular hyperproliferative disorder such as a neoplasia, tumour or cancer, or metastasis. However, a therapeutic benefit or improvement need not be a cure or complete destruction of all target proliferating cells (e.g., neoplasia, tumour or cancer, or metastasis) or ablation of all pathologies, adverse symptoms or complications associated with or caused by cell proliferation or the cellular hyperproliferative disorder such as a neoplasia, tumour or cancer, or metastasis. For example, partial destruction of a tumour or cancer cell mass, or a stabilization of the tumour or cancer mass, size or cell numbers by inhibiting progression or worsening of the tumour or cancer, can reduce mortality and prolong lifespan even if only for a few days, weeks or months, even though a portion or the bulk of the tumour or cancer mass, size or cells remain.

Specific non-limiting examples of therapeutic benefit include a reduction in neoplasia, tumour or cancer, or metastasis volume (size or cell mass) or numbers of cells, inhibiting or preventing an increase in neoplasia, tumour or cancer volume (e.g., stabilizing), slowing or inhibiting neoplasia, tumour or cancer progression, worsening or metastasis, or inhibiting neoplasia, tumour or cancer proliferation, growth or metastasis.

In an embodiment of the invention, administration of the checkpoint inhibitor, selected from a cell, protein, peptide, antibody or antigen binding fragment thereof, directed against CTLA-4, PD-1, PD-L1, PD-L2, B7-H3, B7-H4, B7-H6, A2AR, IDO, TIM-3, BTLA, VISTA, TIGIT, LAG-3, CD40, KIR, CEACAM1, GARP, PS, CSF1R, CD94/NKG2A, TDO, TNFR, DcR3 and combinations thereof, in combination therapy with the non-pathogenic, non-viable Mycobacterium, provides a detectable or measurable improvement or overall response according to the irRC (as derived from time-point response assessments and based on tumour burden), including one of more of the following: (i) irCR—complete disappearance of all lesions, whether measurable or not, and no new lesions (confirmation by a repeat, consecutive assessment no less than 4 weeks from the date first documented), (ii) irPR—decrease in tumour burden 50% relative to baseline (confirmed by a consecutive assessment at least 4 weeks after first documentation). Preferably, the checkpoint inhibitor employed is directed against CTLA-4, PD-1, or PD-L1, and combinations thereof.

In an embodiment of the invention, enhanced therapeutic efficacy is measured by a decrease or stabilisation of tumour size of one or more said tumours, as defined by RECIST 1.1, or iRRC, or iRECIST, or irrRECIST, including stable diseases (SD), a complete response (CR) or partial response (PR) of the target or primary tumour; and/or stable disease (SD) or complete response (CR) of one or more non-target tumours or metastases or micro-metastases.

In an embodiment of the invention, there is provided a method of treating, reducing, inhibiting or controlling a neoplasia, tumour or cancer in a subject, wherein said method comprises:
(i) providing a microneedle device comprising a plurality of microneedles,
(ii) causing the microneedles to penetrate through a stratum corneum into the skin of the subject and optionally assume an anchored state in which the microneedles are anchored in the skin, and project from the microneedle device,
(iii) delivering into the skin via the microneedle device, preferably subcutaneously, subdermally or intradermally, a quantity of a non-pathogenic non-viable Mycobacterium selected from M. vaccae, including the strain deposited under accession number NCTC 11659 and associated designations such as SRL172, SRP299, IMM-201, DAR-901, and the strain as deposited under ATCC 95051 (Vaccae™); M. obuense, M. paragordonae (strain 49061), M. parafortuitum, M. aurum, M. phlei, M. indicus pranii, M.w, M. kyogaense (as deposited under DSM 107316/CECT 9546), M. tuberculosis Aoyama B or H37Rv, and combinations thereof, preferably the strain of Mycobacterium obuense deposited under the Budapest Treaty under accession number NCTC 13365,
wherein said method further comprises simultaneously, separately or sequentially administering to the subject, one or more additional anticancer treatments or agents, wherein said method results in enhanced therapeutic efficacy relative to administration of the one or more checkpoint inhibitors, co-stimulatory checkpoint therapy, one or more additional anticancer treatments or agents, or Mycobacterium alone.

In the preferred embodiment, the subject is not a checkpoint inhibitor refractory patient.ln an embodiment or method of the invention, the one or more additional anticancer treatments or agents is selected from: adoptive cell therapy, surgical therapy, chemotherapy, radiation therapy, hormonal therapy, small molecule therapy such as metformin, receptor kinase inhibitor therapy, hyperthermia treatment, phototherapy, radioablation therapy, anti-angiogenic therapy, cytokine therapy, cryotherapy, biological therapy, HDAC inhibitor e.g. OKI-179, BRAF inhibitor, MEK inhibitor, EGFR inhibitor, VEGF inhibitor, P13K delta inhibitor, PARP inhibitor, mTOR inhibitor, hypomethylating agents, oncolytic virus, TLR agonist including TLR2, 3, 4, 5, 7, 8 or 9 agonists, such as MRx0518 (4D Pharma), STING agonists (including MIW815 and SYNB1891), and cancer vaccines such as GVAX or CIMAvax. In another method of the invention, the anticancer treatment is selected from: microwave irradiation, radiofrequency ablation, targeted radiotherapy such as stereotactic ablative radiotherapy (SABR), embolisation, cryotherapy, ultrasound, high intensity focused ultrasound, cyberknife, hyperthermia, cryoablation, electrotome heating, hot water injection, alcohol injection, embolization, radiation exposure, photodynamic therapy, laser beam irradiation, and combinations thereof.

In an embodiment or method of the invention, the TLR agonists include mifamurtide (Mepact), Krestin (PSK), MRx0518 (4D Pharma), IMO-2125 (tilsotolimod), CMP-001, MGN-1703 (lefitolimod), entolimod, SD-101, GS-9620, imiquimod, resiquimod, MEDI4736, poly I:C such as rintatolimod (Ampligen), CPG7909, DSP-0509, VTX-2337 (motolimod), MEDI9197, NKTR-262, G100 or PF-3512676 and combinations thereof.

Suitable specific combinations include: Ipilimumab+MGN1703, Pembrolizumab+CMP001, Pembrolizumab+SD101, Tremelimumab+PF-3512676, resiquimod+pembolizumab.

In an embodiment or method of the invention, the chemotherapy comprises administration of one or more agents selected from: cyclophosphamide, methotrexate, 5-fluorouracil, doxorubicin, mustine, vincristine, procarbazine, prednisolone, bleomycin, vinblastine, dacarbazine, etoposide, cisplatin, epirubicin, capecitabine, leucovorin, folinic acid, carboplatin, oxaliplatin, gemcitabine, FOLFIRINOX, paclitaxel, nab-paclitaxel (Abraxane), pemetrexed, irinotecan and combinations thereof.

In an embodiment or method of the invention, the one or more additional anticancer treatments or agents is administered intratumorally, intraarterially, intravenously, intravascularly, intrapleurally, intraperitoneally, intratracheally, intranasally, pulmonarily, intrathecally, intramuscularly, endoscopically, intralesionally, percutaneously, subcutaneously, regionally, stereotactically, orally or by direct injection or perfusion.

In a further preferred embodiment or method of the invention, the neoplasia, tumour or cancer is associated with a sarcoma, preferably a soft tissue or non-soft tissue sarcoma. Particularly preferred non-soft tissue sarcomas include bone sarcomas (osteosarcoma, Ewing's sarcoma) and chondrosarcoma. Particularly preferred sarcomas include pleomorphic undifferentiated sarcoma (UPS), angiosarcoma, leiomyosarcoma, dedifferentiated liposarcoma (DDL), synovial sarcoma, rhabdomyosarcoma, epithelioid sarcoma, myxoid liposarcoma, alveolar soft part sarcoma, parachordoma/myoepithelioma, pleomorphic liposarcoma, extraskeletal myxoid chondrosarcoma, or malignant peripheral nerve sheath tumors. The patient may be less than 50 years of age, or less than 20 to 30 years of age, or a teenager or adolescent (<16 years of age), or a child (0 to 14 years of age). Optionally, the one or more sarcoma tumours demonstrate increased staining/expression of PD-L1 or PD-1. Optionally, the non-pathogenic, non-viable Mycobacterium and/or checkpoint inhibitor and/or co-stimulatory binding agent is administered via intratumoral, peritumoral, perilesional or intralesional administration.

Another suitable combination is intratumoral administration of the non-pathogenic non-viable whole cell Mycobacterium together with hafnium oxide nanoparticles, subsequently activated by radiotherapy (NBTXR3, NanoBiotix, Inc.).

In an embodiment of the invention, the combinations and methods disclosed herein result in a clinically relevant improvement in one or more markers of disease status and progression selected from one or more of the following: (i):

overall survival, (ii): progression-free survival, (iii): overall response rate, (iv): reduction in metastatic disease, (v): circulating levels of tumour antigens such as carbohydrate antigen 19.9 (CA19.9), carcinembryonic antigen (CEA), prostate-specific antigen (PSA) or others depending on tumour, (vii) nutritional status (weight, appetite, serum albumin), (viii): systemic immune-inflammation index (SII) or systemic inflammation score (SIS), (ix): pain control or analgesic use, or (x): CRP/albumin ratio or prognostic nutritional index (PNI), or neutrophil/lymphocyte ratio (NLR).

In some embodiments, the one or more markers of disease status and progression selected from the above list may be measured for monitoring of the treatment, reduction, inhibition or control protocols of the present invention. In some preferred embodiments, the one or more biomarkers may include any one or more of: prostate-specific antigen (PSA); carcinoembryonic antigen (CEA); prognostic nutritional index (PNI); systemic immune-inflammation index (SII); or neutrophil/lymphocyte ratio (NLR) and systemic inflammation score (SIS). A therapeutic benefit or beneficial effect is any objective or subjective, transient, temporary, or long-term improvement in the condition or pathology, or a reduction in onset, severity, duration or frequency of an adverse symptom associated with or caused by cell proliferation or a cellular hyperproliferative disorder such as a neoplasia, tumour or cancer, or metastasis. It may lead to improved survival. A satisfactory clinical endpoint of a treatment method in accordance with the invention is achieved, for example, when there is an incremental or a partial reduction in severity, duration or frequency of one or more associated pathologies, adverse symptoms or complications, or inhibition or reversal of one or more of the physiological, biochemical or cellular manifestations or characteristics of cell proliferation or a cellular hyperproliferative disorder such as a neoplasia, tumour or cancer, or metastasis. A therapeutic benefit or improvement therefore may be, but is not limited to destruction of target proliferating cells (e.g., neoplasia, tumour or cancer, or metastasis) or ablation of one or more, most or all pathologies, adverse symptoms or complications associated with or caused by cell proliferation or the cellular hyperproliferative disorder such as a neoplasia, tumour or cancer, or metastasis.

However, a therapeutic benefit or improvement need not be a cure or complete destruction of all target proliferating cells (e.g., neoplasia, tumour or cancer, or metastasis) or ablation of all pathologies, adverse symptoms or complications associated with or caused by cell proliferation or the cellular hyperproliferative disorder such as a neoplasia, tumour or cancer, or metastasis. For example, partial destruction of a tumour or cancer cell mass, or a stabilization of the tumour or cancer mass, size or cell numbers by inhibiting progression or worsening of the tumour or cancer, can reduce mortality and prolong lifespan even if only for a few days, weeks or months, even though a portion or the bulk of the tumour or cancer mass, size or cells remain. Specific non-limiting examples of therapeutic benefit include a reduction in neoplasia, tumour or cancer, or metastasis volume (size or cell mass) or numbers of cells, inhibiting or preventing an increase in neoplasia, tumour or cancer volume (e.g., stabilizing), slowing or inhibiting neoplasia, tumour or cancer progression, worsening or metastasis, or inhibiting neoplasia, tumour or cancer proliferation, growth or metastasis.

In an embodiment of the invention, the combinations, microneedle device, immunomodulator and methods disclosed herein may result in a reduction in incidence, severity and/or duration of injection site reactions, as compared to the same quantity or dose of immunomodulator or Mycobacteria when injected intradermally using a standard syringe and needle, such as a 25G, 26G, 27G, 29G, 30G or 31G needle. Such a reduction may be clinically relevant and enhance future compliance or tolerance to additional injections of said immunomodulator or Mycobacteria. The reduction in incidence, severity and/or duration of said injection site reactions include a reduction in one or more of the following symptoms: pain, tenderness, erythema, swelling/edema, pruritis, haemorrhage, discomfort, induration, anesthesia, discoloration, hematoma, movement impairment, paresthesia, rash, infiltration, ulceration or necrosis. The reduction in incidence, severity and/or duration of said injection site reactions may be observed within 30 minutes, 1 hour, 6, hours, 12 hours or 24 ours or longer after said injection using a microneedle device or method according to the invention.

In an embodiment of the invention, the combinations, microneedle device, immunomodulator and methods disclosed herein may enable the subject to self-administer one or more doses at their home or work or other suitable location.

In another embodiment of the invention, the microneedle device, disclosed herein may have a total pore volume of between 0.001 to 0.2 cubic millimetres, preferably between 0.01 and 0.1 or 0.05 to 0.09 cubic millimetres, whereupon an increase in CD8+ T cells may be observed in the skin or draining lymph node following administration of the immunomodulator or Mycobacteria total pore volume array refers to the maximum volume of the conduits that can be created in the skin after application of a microneedle array.

The term “combination” as used throughout the specification, is meant to encompass the administration of the non-viable Mycobacterium simultaneously, separately or sequentially with administration of another immunomodulator, preferably a checkpoint inhibitor. Accordingly, the checkpoint inhibitor and the non-viable Mycobacterium may be present in the same or separate pharmaceutical formulations, and administered at the same time or at different times.

Dose delays and/ or dose reductions and schedule adjustments are performed as needed depending on individual patient tolerance to treatments.

Alternatively, the administration of a checkpoint inhibitor may be performed simultaneously with the administration of the effective amounts of the Mycobacterium.

The non-pathogenic, non-viable Mycobacterium, may be administered to the patient via the parenteral, intratumoral, oral, sublingual, nasal or pulmonary route via a miconeedle device according to the invention. In a preferred embodiment, it is administered via a parenteral route selected from subcutaneous, intradermal, subdermal, and intravesicular administration. In other embodiments, administration comprises intratumoural delivery of the mycobacterial preparation.

In an embodiment of the invention, the effective amount of the Mycobacterium may be administered as a single dose. Alternatively, the effective amount of the Mycobacterium may be administered in multiple (repeat) doses, for example two or more, three or more, four or more, five or more, ten or more, or twenty or more repeat doses. The Mycobacterium may be administered between about 4 weeks and about 1 day prior to checkpoint inhibitor therapy, such as between about 4 weeks and 1 week, or about between 3 weeks and 1 week, or about between 3 weeks and 2 weeks. Administration may be presented in single or multiple doses.

Mycobacterial compositions according to the invention will comprise an effective amount of mycobacteria typically dispersed in a pharmaceutically acceptable carrier. The phrases “pharmaceutically or pharmacologically acceptable” refers to molecular entities and compositions that do not produce an adverse, allergic or other untoward reaction when administered to an animal, such as, for example, a human, as appropriate. The preparation of a pharmaceutical composition that contains mycobacteria will be known to those of skill in the art in light of the present disclosure, as exemplified by Remington's Pharmaceutical Sciences, 18th Ed. Mack Printing Company, 1990, Moreover, for animal (e.g., human) administration, it will be understood that preparations should meet sterility, pyrogenicity, general safety and purity standards. A specific example of a pharmacologically acceptable carrier as described herein is borate buffer or sterile saline solution (0.9% NaCl).

As used herein, “pharmaceutically acceptable carrier” includes any and all solvents, dispersion media, coatings, surfactants, antioxidants, preservatives e.g., antibacterial agents, antifungal agents), isotonic agents, absorption delaying agents, salts, preservatives, drugs, drug stabilizers, gels, binders, excipients, disintegration agents, lubricants, sweetening agents, flavouring agents, dyes, such like materials and combinations thereof, as would be known to one of ordinary skill in the art (see, for example, Remington's Pharmaceutical Sciences, 18th Ed. Mack Printing Company, 1990, pp. 1289-1329).

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine study, numerous equivalents to the specific procedures described herein. Such equivalents are considered to be within the scope of this invention and are covered by the claims. All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference. The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects. As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

The application of the microneedle technology disclosed herein, is preferably not intended to be used to treat a subject characterised as checkpoint inhibitor refractory. As used herein, the term “checkpoint inhibitor refractory” refers to a subject/patient identified as non-responsive to checkpoint inhibitor therapy. A refractory subject/patient may exhibit an innate (primary) resistance to checkpoint inhibitor therapy. Innate resistance may be demonstrated by lack of response or an insufficient response to said checkpoint inhibitor therapy for at least about 8 weeks, or 12 weeks from the first date. A refractory patient may exhibit an acquired (secondary) resistance to checkpoint inhibitor therapy. Acquired resistance may be demonstrated by an initial response to said checkpoint therapy but with a subsequent relapse and progression of one or more tumours. Checkpoint inhibitor refractory patients can be non-responsive to any checkpoint inhibitor, non-limiting examples of which include CTLA-4, PD-1, PD-L1, PD-L2, B7-H3, B7-H4, B7-H6, A2AR, IDO, TIM-3, BTLA, VISTA, LAG-3 inhibitors or combinations thereof.

All documents referred to herein are incorporated by reference to the fullest extent permissible.

Any element of a disclosure is explicitly contemplated in combination with any other element of a disclosure, unless otherwise apparent from the context of the application.

Having now generally described the invention, the same will be more readily understood through reference to the following examples which are provided by way of illustration, and are not intended to be limiting of the present invention, unless specified.

### EXAMPLE 1

The present invention may be carried out in the following exemplary way: A suspension comprising 0.1 mg M. obuense NTCT 13365 in borate buffer, can be mixed into a 12.5% CMC hydrogel solution and the applied to a casting mold assembly, with a layer of blank CMC solution applied on top, to form a solid microneedle device deploying a 10×10 array of microneedles of 350 microns in length. A more concentrated suspension, using 1 mg M. obuense NCTC 13365 in borate buffer, can also be used.

### EXAMPLE 2

The present invention may be carried out in the following exemplary way: A suspension comprising 1 mg M. obuense NCTC 13365 in 0.1 ml borate buffer, can be presented in a suitable syringe with a MicronJet600 (NanoPass, Israel) microneedle device attached to the open end of said syringe, then applied to the skin of a subject and injected at a steady and slow rate. Such a procedure can result in a reduced site reaction.

### EXAMPLE 3

Microneedles can be prepared from aqueous blends of Gantrez® S-97 (methyl vinyl ether and maleic acid copolymers). A stock of Gantrez® S-97 (40%) is diluted with the appropriate volume of a suspension of M. obuense NCTC 13365 diluted in borate buffered saline (PBS), pH 8.2, to give a final concentration of 20% Gantrez® S-97. One hundred milligrams of this suspension, containing 200 μg of heat-inactivated M. obuense is poured onto silicon micromoulds. The micromoulds contain 361 (19×19) pyramidal shaped needles, perpendicular to the base, 500 μm in height, 300 μm wide and with 50 μm interspacing. The arrays have an area of 0.6 sq. cm. A pressure of 3 bar is subsequently used to force the formulation into the needles of the miocroneedle mould. The microneedles are dried under controlled temperature conditions (approx. 19° C.) for about 24 hours, before being carefully removed from the moulds and stored until further use.

1-56. (canceled)

57. A microneedle device comprising a plurality of microneedles, and contained thereon or therein a composition comprising a non-pathogenic, non-viable Mycobacterium.

58. A kit of parts for delivering a non-pathogenic, non-viable Mycobacterium into the skin of a subject, comprising:
a microneedle device comprising a plurality of microneedles, and a non-pathogenic non-viable Mycobacterium is selected from M. vaccae, M. vaccae deposited under NCTC 11659, M. vaccae deposited under ATCC 95051, M. obuense, M. obuense deposited under NCTC 13365, M. paragordonae (strain 49061), M. parafortuitum, M. aurum, M. phlei, M. indicus pranii, M.w, M. kyogaense deposited under DSM 107316/CECT 9546, M. tuberculosis Aoyama B or H37Rv, and combinations thereof; and
one or more biologically-active agents selected from a therapeutic drug, nutraceutical, vaccine, cell, virus, lysate, vector, gene, mRNA, DNA, nucleic acid, protein, polypeptide, peptide, antibody, bispecific antibody, multi-specific antibody, ADC (antibody-drug conjugate), Fab fragment (Fab), F(ab')2 fragment, diabody, triabody, tetrabody, probody, single-chain variable region fragment (scFv), disulfide-stabilized variable region fragment (dsFv), or other antigen binding fragment thereof, optionally wherein said biologically-active agent is an antigen or antigenic determinant.

59. The kit of parts according to claim 58, wherein said one or more biologically-active agents is a checkpoint inhibitor, selected from a cell, protein, peptide, antibody or antigen binding fragment thereof, directed against CTLA-4, PD-1, PD-L1, PD-L2, LAG-3, B7-H3, B7-H4, B7-H6, A2AR, IDO, TIM-3, BTLA, VISTA, TIGIT, LAG-3, CD40, KIR, CEACAM1, GARP, PS, CSF1R, CD94/NKG2A, TDO, TNFR, DcR3, CD27, CD28, CD40, CD122, CD137, 0X40, GITR, ICOS and combinations thereof.

60. A method of treating, reducing, inhibiting or controlling a neoplasia, tumour or cancer in a subject, and wherein said method comprises:
(i) providing a microneedle device comprising a plurality of microneedles,
(ii) causing the microneedles to penetrate through a stratum corneum into the skin of the subject and optionally assume an anchored state in which the microneedles are anchored in the skin, and project from the microneedle device,
(iii) delivering into the skin via the microneedle device a quantity of a non-pathogenic non-viable Mycobacterium selected from M. vaccae, M. vaccae deposited under NCTC 11659, M. vaccae deposited under ATCC 95051, M. obuense, M. paragordonae (strain 49061), M. parafortuitum, M. aurum, M. phlei, M. indicus pranii, M.w, M. kyogaense deposited under DSM 107316/CECT 9546, M. tuberculosis Aoyama B or H37Rv, and combinations thereof.

61. The method according to claim 60, wherein said subject to be treated is not a checkpoint inhibitor refractory patient.

62. The method according to claim 60, wherein said method further comprises simultaneously, separately or sequentially administering to the subject, one or more biologically-active agents selected from a therapeutic drug, nutraceutical, vaccine, cell, virus, lysate, vector, gene, mRNA, DNA, nucleic acid, protein, polypeptide, peptide, antibody, bispecific antibody, multi-specific antibody, ADC (antibody-drug conjugate), Fab fragment (Fab), F(ab')₂ fragment, diabody, triabody, tetrabody, probody, single-chain variable region fragment (scFv), disulfide-stabilized variable region fragment (dsFv), or other antigen binding fragment thereof, optionally wherein said biologically-active agent is an antigen or antigenic determinant.

63. The method according to claim 60, wherein the one or more biologically-active agents is a checkpoint inhibitor directed against CTLA-4, PD-1, PD-L1, PD-L2, B7-H3, B7-H4, B7-H6, A2AR, DO, TIM-3, BTLA, VISTA, TIGIT, LAG-3, CD40, KIR, CEACAM1, GARP, PS, CSF1R, CD94/NKG2A, TDO, TNFR, DcR3 and combinations thereof.

64. The method according to claim 60, wherein the one or more biologically-active agents is selected from: thymosin or a purified fraction thereof, an HDAC inhibitor, BRAF inhibitor, MEK inhibitor, EGFR inhibitor, VEGF inhibitor, P13K delta inhibitor, PARP inhibitor, mTOR inhibitor, hypomethylating agents, oncolytic virus, TLR agonists, STING agonists, cancer vaccines, and combinations thereof.

65. The method according to claim 60, wherein said one or more biologically-active agents are administered at the same time and/or via the same microneedle device as the non-pathogenic non-viable Mycobacterium, or at separate times and/or via separate routes of administration.

66. The method according to claim 60, wherein said non-viable Mycobacterium is a rough variant and/or whole cell.

67. The method according to claim 60, wherein said non-pathogenic, non-viable Mycobacterium is presented as a lysate, homogenate or sonicate of a whole cell, or fractions thereof.

68. The method according to claim 60, wherein said non-pathogenic, non-viable Mycobacterium is presented as a suspension or in dehydrated form.

69. The method according to claim 60, wherein said neoplasia, tumour or cancer is associated with a cancer selected from prostate cancer, liver cancer, renal cancer, lung cancer, breast cancer, colorectal cancer, breast cancer, pancreatic cancer, brain cancer, glioblastoma, hepatocellular cancer, lymphoma, leukaemia, gastric cancer, cervical cancer, ovarian cancer, thyroid cancer, head and neck cancer, skin cancer, melanoma, or a sarcoma.

70. The method according to claim 69, wherein said neoplasia, tumour or cancer is associated with a cancer selected from pancreatic, colorectal, prostate, gastro-oesophageal, skin, breast, brain, glioblastoma, melanoma, sarcoma or ovarian, optionally wherein said cancer is locally advanced, inoperable, borderline operable, or resectable.

71. The method according to claim 60, wherein the neoplasia, tumour or cancer is metastatic, disseminated or micrometastatic.

72. The method according to claim 60, wherein the amount of non-pathogenic non-viable Mycobacterium administered is a therapeutically effective amount.

73. The method according to claim 60, wherein said method results in enhanced therapeutic efficacy relative to administration of the one or more biologically-active agents or non-viable w hole cell Mycobacterium alone.

74. The method according to claim 73, wherein said enhanced therapeutic efficacy is:
a) measured by increased overall survival time;
b) measured by increased progression-free survival;
c) measured by a decrease or stabilisation of tumour size of one or more said tumours, as defined by RECIST 1.1, or iRRC, or iRECIST, or irrRECIST, stable disease (SD), a complete response (CR) or partial response (PR) of the target or primary tumour; and/or stable disease (SD) or complete response (CR) of one or more non-target tumours or metastases or micro-metastases; or
d) measured by an improved overall response rate and/or increased quality of life.

75. The method according to claim 60, wherein said method results in a reduction in incidence, severity and/or duration of injection site reactions, as compared to the same quantity or dose of immunomodulator or Mycobacteria when injected intradermally using a standard syringe and needle.
